(19) United States
(12) Patent Application Publication
(10) Pub. No.: US 2025/0009531 A1
(43) Pub. Date: Jan. 9, 2025

(54) METHOD FOR GENERATING ACOUSTIC FEEDBACK, AND ORTHOPAEDIC DEVICE

(71) Applicant: OTTO BOCK HEALTHCARE PRODUCTS GMBH, Wien (AT)

(72) Inventors: Josef INSCHLAG, Wien (AT); Marcus EDER, Wien (AT)

(21) Appl. No.: 18/710,918

(22) PCT Filed: Dec. 1, 2022
(86) PCT No.: PCT/EP2022/084045

§ 371 Date: May 16, 2024

(30) Foreign Application Priority Data
Dec. 7, 2021 (DE) 10 2021 132 188.3

(51) Int. Cl. A61F 2/70; A61F 2/54; A61F 2/60; A61F 2/68

(52) U.S. Cl. CPC A61F 2/70; A61F 2/54; A61F 2/60; A61F 2002/546; A61F 2002/607; A61F 2002/6809; A61F 2002/701

(57) ABSTRACT

The invention relates to a method for generating acoustic feedback of an orthopaedic device comprising at least one electric motor (13), which has a stator (30) and a rotor (40) which can rotate about a rotational axis (50) and is coupled to a component of the orthopaedic device, which component can be moved by the electric motor (13), wherein the electric motor (13) is oscillatingly operated by repeated pole changing of at least one motor voltage, without the rotor (4) performing a complete rotation about the rotational axis (50).

The invention relates to a method for generating acoustic feedback of an orthopedic device comprising at least one motor, which has a control device, a stator and a rotor which can rotate about an axis of rotation and is coupled to a component of the orthopedic device which can be adjusted by the motor, and to an orthopedic device of this type.

Acoustic feedback is used in orthopedic devices to communicate certain information to the user, for example when a particular movement is performed, a particular grip force is reached, an object is touched or a movement or grip pattern has been completed. In this way, acoustic feedback makes it easier for the user to deal with the orthopedic device and offers them security in doing so.

EP 3 753 536 A1 discloses a feedback system for a prosthesis comprising a glove, a sensor which is arranged on the glove and which generates a measurement signal, and at least two complementary feedback devices which convert the measurement signal into a feedback signal. The feedback devices may be designed as a light element, as a vibration motor, as a pressure actuator or as a loudspeaker.

Sometimes acoustic feedback is provided to orthopedic devices by separate loudspeakers, vibration motors or pressure actuators. This results in additional costs for the provision of the components, which also take up part of the limited installation space.

The object of the present invention is to provide a method and a device for generating acoustic feedback that are cost-effective and space-saving.

According to the invention, this object is achieved by a method having the features of the main claim and an orthopedic device having the features of the additional independent claim. Advantageous embodiments and developments of the invention are disclosed in the dependent claims, the description and the figures.

The method for generating acoustic feedback of an orthopedic device comprising at least one electric motor, which has a stator and a rotor which can rotate about an axis of rotation and is coupled to a component of the orthopedic device which can be adjusted by the electric motor makes provision for the motor to be operated in an oscillating manner by repeated reversal of the polarity of at least one motor voltage, without the rotor performing a complete rotation about the axis of rotation. The movement of the rotor causes an acoustic noise which can be used as a signal for the user of an orthopedic device. Since the repeated, in particular periodic, reversal of the polarity is an oscillating movement, the rotor performs only small oscillations or rotational movements about the axis of rotation thereof, with the result that a component of the orthopedic device which is coupled to the electric motor is not moved or is moved only minimally.

One development makes provision for a square-wave voltage, delta voltage, sawtooth voltage, sinusoidal curve voltage or a mixed form thereof to used as the motor voltage. Such waveforms are used as a pattern for the polarity reversal, which pattern can be repeated several times when generating an acoustic noise. The respective waveform in this case significantly influences the timbre of the generated acoustic feedback.

The polarity reversal is preferably operated at a frequency between 20 Hz and 6000 Hz, but may also go beyond this and reach the human hearing threshold. A higher polarity reversal frequency generates acoustic feedback at a higher frequency. In the case of a periodic polarity reversal, the pitch of the acoustic feedback depends on the frequency of the polarity reversal.

Advantageously, an electric motor with different polarity reversal frequencies is operated in a time sequence and/or several electric motors with different polarity reversal frequencies are operated simultaneously or successively. In this way, it is possible on the one hand to produce a sequence of several tones and/or sounds; on the other hand, polyphonic sounds can be produced by the simultaneous use of several electric motors.

The amplitude of the motor voltage is preferably changed during the polarity reversal. If the amplitude of the motor voltage is changed during the polarity reversal, the volume of the acoustic feedback is affected. This allows tones and sounds to be produced with different volumes or with increasing or decreasing volumes.

One development makes provision for the motor to be designed as a commutator motor and for the polarity of the motor voltage of the electric motor to be reversed repeatedly, in particular periodically, to generate an acoustically perceptible signal. Commutator motors are inexpensive to manufacture and do not require a complex control system. Commutator motors usually produce louder noises than brushless DC motors due to the friction of the sliding contacts on the commutator.

One variant makes provision for the motor to be designed as a brushless DC motor and for the phase of the rotating field of the electric motor to be repeated, in particular changed periodically, to generate an acoustically perceptible signal. Brushless DC motors generate a low amount of heat and therefore have longer service lives. In addition, brushless DC motors are characterized by high efficiency and are therefore generally smaller than commutator motors at the same power.

The rotor is advantageously moved in an oscillating manner by an angle of rotation of not more than 120°, in particular not more than 90°, to generate an acoustically perceptible signal. In common drive trains of components of an orthopedic device, such angles of rotation only lead to a minimal movement, or no movement at all, of the respective driven component, such that no significant unwanted movement is carried out when generating an acoustically perceptible signal. The drive train components comprise, for example, a gear or a mechanical clearance, so that usually only the drive train components are moved, but not the driven component of the orthopedic device, in the case small angles of rotation. The motor moves, but only very minimally. The higher the frequency of the polarity reversal, the smaller the movement of the rotor. Due to inertia, the motor is less and less able to follow the changes in direction at higher frequencies. However, a minimal movement of the rotor is necessary to generate an audible signal. The amplitude of the rotor oscillation is comparable to that of a piezo loudspeaker. The lower the frequency, the louder the signal.

The orthopedic device comprising at least one electric motor, which has a stator and a rotor which can rotate about an axis of rotation and is coupled to a component of the orthopedic device which can be adjusted by the electric motor and is connected to a control device, makes provision for the control device to be set up to repeatedly reverse the polarity of at least one motor voltage of the electric motor to generate an acoustically perceptible signal, without the rotor performing a complete rotation about the axis of rotation. Due to the fact that an electric motor of an adjustable component of the orthopedic device is used to generate an acoustically perceptible signal, no further loudspeakers, other devices for generating acoustic feedback or separate electric motors for generating the acoustic feedback are necessary. On the one hand, this may reduce the costs of the orthopedic device. On the other hand, the installation space for the separate components otherwise necessary for generating acoustic feedback is no longer available and is thus available for other components or for reducing the size of the orthopedic device. Since the rotor of the electric motor only performs an incomplete rotation about the axis of rotation when generating the acoustic feedback, the component of the orthopedic device which can be adjusted by the electric motor is moved only minimally or not at all, such that no disadvantages arise with regard to the operation by the user.

The control device is advantageously set up to generate a polarity reversal frequency between 20 Hz and 6000 Hz or more. Polarity reversal frequencies from this range can be used to achieve sufficient volumes for acoustic feedback. The pitches are in the frequency range of human hearing. At the same time, the required voltage amplitudes in this reverse polarity frequency range are relatively low.

Different voltage amplitudes are preferably present at different polarity reversal frequencies. By using different frequency-dependent voltage amplitudes, it is possible to make the maximum rotation constant even at different frequencies.

One development makes provision for the electric motor to be designed as a brushless DC motor or commutator motor. Commutator motors are easy to manufacture on account of their simple design, whereas brushless DC motors are characterized by high efficiency.

An exemplary embodiment of the invention is explained in more detail below on the basis of

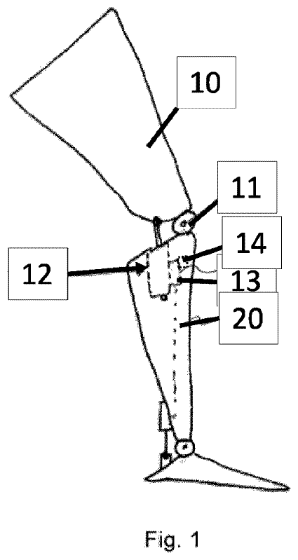

FIGS. 1 to 3. The same reference signs refer to the same components. In the figures:

FIG. 1—shows a schematic illustration of an orthopedic device;

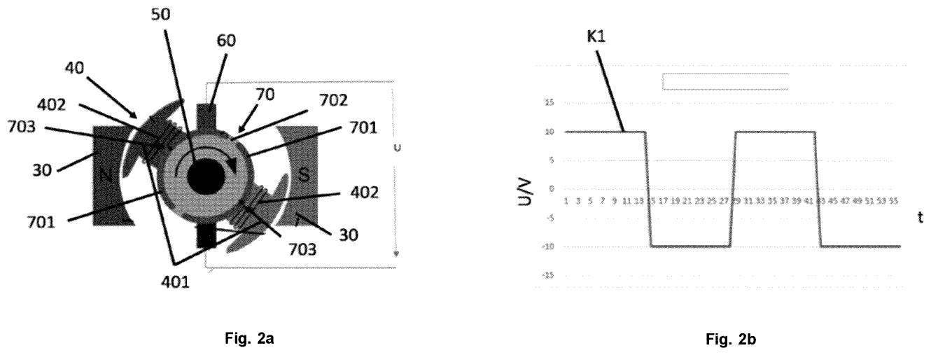

FIG. 2a—shows a commutator motor;

FIG. 2b—shows a graph of the motor voltage over time;

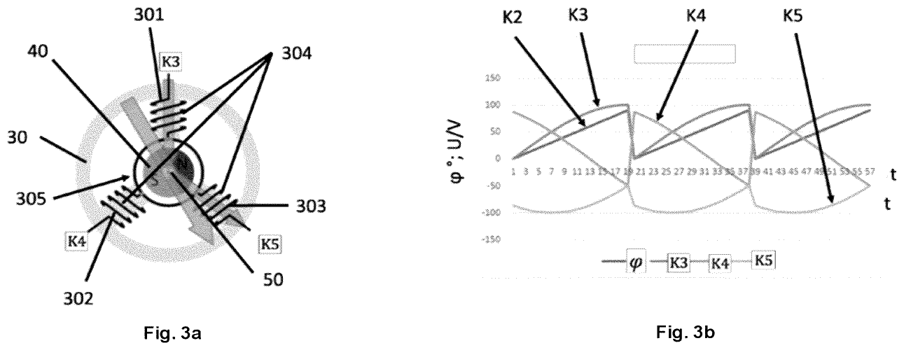

FIG. 3a—shows a brushless DC motor;

FIG. 3b—shows a graph of the motor angle and the pole voltages over time; and

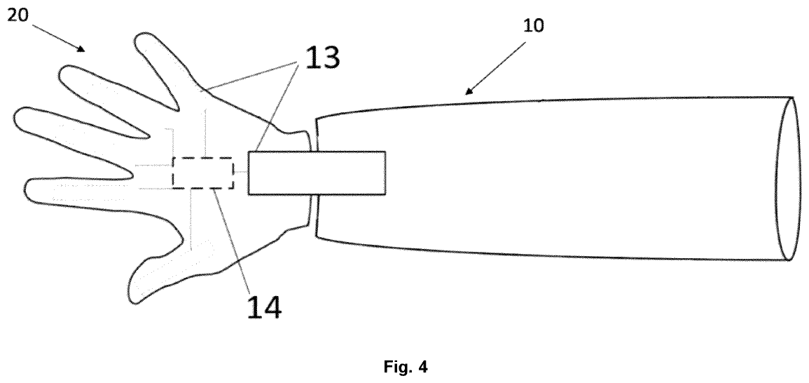

FIG. 4—shows a schematic illustration of an upper limb prosthesis.

FIG. 1 shows a schematic illustration of an orthopedic device in the form of a lower limb prosthesis comprising an upper part 10 and a lower part 20, which parts are mounted on one another in an articulated manner about a pivot axis 11. In the exemplary embodiment illustrated, the upper part 10 has a prosthetic shaft which is used to receive a thigh stump. Other orthopedic devices such as upper limb prostheses and upper and lower limb orthoses are further examples of an orthopedic device. An actuator 12 is arranged between the upper part 10 and the lower part 20. In the exemplary embodiment illustrated, the actuator 12 is designed as a linearly acting actuator 12 and comprises a drive 13 in the form of an electric motor to adjust the position of the upper part 10 relative to the lower part 20. The actuator 12 may be formed, for example, as a hydraulically acting actuator and comprise a pump which is driven by the drive 13. A hydraulic fluid is pumped via the pump into a pump chamber or into a chamber closed by a piston. The piston is coupled to a piston rod mounted on the upper part 10 or lower part 20. By pumping the hydraulic fluid appropriately into the pump chamber, the piston rod is moved in one direction or another and the upper part 10 is pivoted relative to the lower part 20. This makes it possible to carry out, brake or support an extension movement or a flexion movement of the orthopedic device. As an alternative to a hydraulic embodiment of the actuator 12, it may also be of mechanical design, such that a spindle is driven via the drive 13, for example. The spindle can then be extended into and retracted from an actuator housing to bring about an extension or flexion of the orthopedic device. A control device 14 is arranged on the drive 13, the control device allowing repeated reversal of the polarity of a motor voltage of the drive 13, such that the drive carries out an oscillating movement using which an acoustic feedback is generated.

FIG. 2a shows a commutator motor. The commutator motor comprises a stator 30 which is designed as a permanent magnet with a magnetic north pole N and a magnetic south pole S. The rotor 40 is arranged between the two magnetic poles. The rotor 40 is pivotable about an axis of rotation 50 and, in the exemplary embodiment shown, is designed as a double T-rotor with two T-shaped head elements 401, which are arranged symmetrically along an axis perpendicular to the axis of rotation 50. A cylindrical commutator 70 is arranged on the rotor 40 concentrically to the axis of rotation 50, the rotor being permanently connected to the commutator. Two opposite sides of the commutator 70 have sliding contacts 60 arranged on them. Two conductive zones 701 are arranged on the outer surface of the commutator 70. The two conductive zones 701 are spatially separated from one another by way of a non-conductive zone 702. The non-conductive zone 702 extends on two opposite areas to the outer surface of the commutator 70 and thus divides the conductive zone 701 into two areas of equal size. The conductive zones 701 are each connected via a connection 703 to the rotor windings 402 which are arranged on the cylindrical portions of the two T-shaped head elements 401. In the position shown, the sliding contacts 60 make contact with the conductive zones 701. Since a motor voltage U is applied to the sliding contacts 60, an electromagnetic field is generated at the rotor via the rotor winding 402. The rotor 40 begins to rotate due to the attraction and repulsion from the permanent magnet of the stator 30. During rotation, the commutator 70 is used as a mechanical switch for reversing the polarity of the electromagnetic field at the rotor 40, such that continuous rotation is possible. A control device 14 can be used to reverse the polarity of the motor voltage U applied to the sliding contacts 60. Such a reversal of the polarity results in the rotor 40 forming an electromagnet with the opposite polarity. This causes the rotor 40 to rotate in the opposite direction. If the polarity reversal is repeated by the control device 14, in particular periodically, the rotor 40 performs an oscillating movement which can be used to generate acoustic feedback.

FIG. 2b shows a graph of the motor voltage U at a commutator motor over time. The curve K1 shows a square-wave curve of the motor voltage U over time t. At the beginning, the motor voltage is 10 volts, after 14 time units it changes abruptly to −10 volts, after another 14 time units the motor voltage jumps again to 10 volts. The polarity is reversed periodically. Aperiodic polarity reversals are also possible. A square-wave form is used here as a waveform for the polarity reversal. It is also possible to use delta, sawtooth or sinusoidal waveforms or a mixture of these. The frequency of the waveform illustrated is about 36 Hz at a time unit of t in milliseconds; other frequencies, in particular several superimposed frequencies, which lie in the hearing range of the human ear, are also conceivable.

FIG. 3a shows a brushless DC motor. In the brushless DC motor, the rotor 40 is designed as a cylindrical permanent magnet which is located concentrically within the stator 30, has a north pole N and a south pole S, and is pivotable about the axis of rotation 50. The stator 30 is designed as a cylindrical sleeve which has three inwardly projecting, cylindrical, ferromagnetic cores 301-303, on each of which a winding 304 is arranged. The cores 301-303 are arranged at an angle of 120° with respect to one another. The windings 304 of the three cores 301-303 are connected to one another via a circuit system which is not shown in any more detail. Independent motor voltages U, V, W are applied to the various windings 304 via the circuit system, such that independent electromagnets are created at the three cores 301-303. A suitable reversal of the polarity of the motor voltages over time can cause the rotor 40 to rotate about the axis of rotation 50. For example, U, V, and W are phase-shifted, sinusoidal AC voltages. If the AC voltage at the second core 302 is shifted by 120° and the one at the third core 303 by 240° in comparison with the first core 301, the rotor 40 is rotated about the axis of rotation 50 constantly in synchronization with the AC voltage. Only two or more than three cores can also be arranged on the stator 30.

FIG. 3b shows a graph of the rotor angle φ and the motor voltages U, V, W at a brushless DC motor over time. The curve K2 shows the course of the rotor angle φ over time. The rotor angle φ starts at 0° and first increases linearly up to a value of 90° and then drops abruptly back to the initial value of 0°. This process then starts again. The curves K3, K4 and K5 show the courses of the motor voltages U, V, W over time. The motor voltages U, V, W start at different initial values and first show the course of three sinusoidal curves, each phase-shifted by 120°, with an amplitude of 100 V. The first motor voltage U increases, for example, in the time window in which the rotor angle increases to 90°, sinusoidally up to the maximum value of 100 V. In order to rapidly change the rotor angle φ to the initial value of 0°, the polarity of the motor voltages U, V, W are also reversed to their respective initial value. This is only possible because the rotor 40 has only performed a small rotation. If the rotor 40 has performed a rotation of more than one rotation, it cannot reverse this rotation by a sudden reversal of the polarity, but can only be moved to its relative initial position, however, in which it has performed a rotation of 360° or an integer multiple of thereof compared to the initial position.

Irrespective of the design of the electric motor 13, it is possible, through the repeated, in particular periodic, reversal of the polarity of at least one motor voltage, to operate the electric motor 13 in an oscillating manner, without the rotor 40 performing a complete rotation about the axis of rotation 50. Advantageously, the polarity is reversed in such a way that none of the components of the orthopedic device are moved. Due to the manufacturing tolerances, the necessary clearance and the gear units or transmission devices necessarily arranged between the electric motor 13 and the common components, the rotor 40 can implement comparatively large angles of rotation of less than 120°, without a relative displacement of the components taking place. In particular, the angle of rotation of less than 90° is set, whereby the rotor 40, depending on frequency and mechanics, almost does not move at all, but only generates a torque which periodically changes the sign. The movement or the change in torque is sufficient to generate acoustic feedback that the user of the orthopedic device can perceive. The acoustic perceptible signal is advantageously in a frequency range between 20 Hz and 6000 Hz.

FIG. 4 illustrates a further embodiment of the orthopedic device in the form of an upper limb prosthesis. The orthopedic device has a prosthetic shaft as upper part 10 for receiving a forearm stump and a prosthetic hand arranged distally thereto as lower part 20. Several electric motors 13 are arranged within the prosthetic hand 20, an electric motor 13 is used to move the prosthetic hand 20 relative to the prosthesis shaft 10, while several electric motors 13 are arranged within the prosthetic hand 20 which move the fingers or the thumb relative to a main body of the prosthetic hand 20 or relative to a chassis. All electric motors 13 are coupled to a control device 14 via which it is possible to reverse the polarity of the voltage of respective motors 13 repeatedly, such that the respective electric motor 13 oscillates, without the rotor of the electric motor 13 performing a complete rotation about the axis of rotation thereof.

### LIST OF REFERENCE SIGNS

10 Upper part
11 Pivot axis
12 Actuator
13 Drive
14 Control device
20 Lower part
30 Stator
301 First core
302 Second core
303 Third core
304 Winding
305 Circuit system
40 Rotor
401 Head elements
402 Rotor winding
50 Axis of rotation
60 Sliding contact
70 Commutator
701 Conductive zone
702 Non-conductive zone
K1 Motor voltage over time
K2 Rotor angle over time
K3 First motor voltage over time
K4 Second motor voltage over time
K5 Third motor voltage over time
N Magnetic north pole
S Magnetic south pole
U Motor voltage
V Second motor voltage
W Third motor voltage
φ Angle of the rotor

1. A method for generating acoustic feedback of an orthopedic device comprising at least one electric motor comprising a stator and a rotor configured to rotate about an axis of rotation and wherein the rotor is coupled to a component of the orthopedic device which is adjustable by the electric motor, the method comprising the steps of:
operating the at least one electric motor in an oscillating manner by repeated reversal of a polarity of at least one motor voltage, and
rotating the rotor about the axis of rotation but without the rotor performing a complete rotation about the axis of rotation.

2. The method as claimed in claim 1, wherein a square-wave voltage, delta voltage, sawtooth voltage, sinusoidal curve voltage or a mixed form thereof is used as the at least one motor voltage during the operating step.

3. The method as claimed in claim 1 wherein the repeated reversal of the polarity of the at least one motor voltage is operated at a frequency ranging from 20 Hz to 6000 Hz.

4. The method as claimed in claim 1 wherein the at least one electric motor is operated with different polarity reversal frequencies in a time sequence and/or wherein the at least one electric motor comprises several electric motors with different polarity reversal frequencies and the several electric motors are operated simultaneously or successively.

5. The method as claimed in claim 1 further comprising changing an amplitude of the at least one motor voltage during the polarity reversal.

6. The method as claimed in claim 1 wherein the at least one electric a motor is a commutator motor, and further comprising the step of periodically reversing the polarity of the at least one motor voltage of the at least one electric motor to generate an acoustically perceptible signal.

7. The method as claimed in claim 1 wherein the at least one electric motor is a brushless DC motor, and further comprising the step of periodically changing a phase of a rotating field of the brushless DC motor to generate an acoustically perceptible signal.

8. The method as claimed in claim 1 wherein the rotor is moved in an oscillating manner by an angle of rotation of not more than 120° to generate an acoustically perceptible signal.

9. An orthopedic device, comprising:
at least one electric motor comprising a stator and a rotor, wherein the rotor is rotatable an axis of rotation, and wherein the rotor is coupled to a component of the orthopedic device which is adjustable by the at least one electric motor;
a control device connected to the at least one electric motor, wherein the control device is set up to repeatedly reverse a polarity of at least one motor voltage of the at least one electric motor to generate an acoustically perceptible signal, without the rotor performing a complete rotation about the axis of rotation.

10. The orthopedic device as claimed in claim 9, wherein the control device is set up to generate a polarity reversal frequency ranging from 20 Hz to 6000 Hz.

11. The orthopedic device as claimed in claim 9 wherein the control device and/or the at least one electric motor is configured such that different voltage amplitudes are present at different polarity reversal frequencies.

12. The orthopedic device as claimed in claim 9 wherein the at least one electric motor is a brushless DC motor or commutator motor.

13. The method of claim 8 wherein the angle of rotation is not more than 90°.
